(19) United States
(12) Patent Application Publication
(10) Pub. No.: US 2019/0377051 A1
(43) Pub. Date: Dec. 12, 2019

(54) GENERATING A MOVEMENT SIGNAL OF AN OBJECT

(71) Applicant: Siemens Healthcare GmbH, Erlangen (DE)

(72) Inventors: Mario Bacher, Erlangen (DE); Peter Speier, Erlangen (DE)

(21) Appl. No.: 16/435,340

(22) Filed: Jun. 7, 2019

(30) Foreign Application Priority Data
Jun. 8, 2018 (EP) 18176780.7

(51) Int. Cl. G01R 33/565; G01R 33/567; A61B 5/055; A61B 5/00

(52) U.S. Cl. CPC G01R 33/56509; A61B 5/0033; A61B 5/055; G01R 33/5676

(57) ABSTRACT

A method for generating a movement signal of an object such as a body part of a human or animal is provided. The movement signal provides quantitative information on a movement of the object. The method includes acquiring an electromagnetic navigation signal such as a Pilot Tone signal from the object. The electromagnetic navigation signal is modulated by movements of the object. A reference signal is extracted from the navigation signal, and a parameter having a known time-dependency is determined from the reference signal. The navigation signal is corrected based on the parameter or a time-average of the parameter to reduce a signal drift in the navigation signal. The movement signal is extracted from the corrected navigation signal.

This application claims the benefit of EP 18176780.7, filed on Jun. 8, 2018, which is hereby incorporated by reference in its entirety.

### BACKGROUND

The present embodiments relate to generating a movement signal of an object.

Many medical imaging modalities, such as computer tomography (CT), magnetic resonance imaging (MRI) or positron emission tomography (PET) are sensitive to patient motion during image acquisition. For example, high quality and high coverage MRI measurements typically take several minutes. During this time, motion inside the imaged volume will degrade image quality and make the image non-diagnostic unless the motion is tracked and taken into account during reconstruction. Apart from motion in non-compliant patients, the unavoidable respiratory motion and cardiac motion is to be taken into account.

Exemplary devices and methods for motion correction are described in US 20170160367 A1, US 20160313432 A1, US 20100142789 A1, US 20140355855 A1, and U.S. Pat. No. 7,205,763 B2.

According to the prior art, motion artifacts in medical images are reduced by triggering the image acquisition to extreme motion states, like end-inspiration or end-expiration. This suffices if the respiratory motion is regular. However, in clinical practice, respiratory patterns (e.g., the position of end-inspiration) often change during an examination. To achieve robust motion synchronization, therefore, quantitative navigation methods are desired (e.g., methods providing more information about the motion than the times of the extreme motion states).

An example of quantitative navigation in MRI is respiratory gating using liver dome MR navigators. Here, a specific navigator value corresponds to one geometric respiratory phase for the whole scan. Thus, the method assumes that respiration may be described by a single parameter (e.g., the position of the upper side of the liver).

Electromagnetic navigation (EMN) methods like the pilot tone (PT) method or the Biomatrix respiratory sensor have recently been investigated for providing motion information. However, these methods tend to be affected by signal drifts. This is because the modulation depth due to the motion to be observed is at best of the order of a few percent. Thus, minor drifts in the movement signal (e.g., caused by minor gain changes in receive path electronics) create large drifts in the net motion signal.

The pilot tone navigation method has first been described, for example, in Lea Schroder, Jens Wetzel, Andreas Maier, Lars Lauer, Jan Bollenbeck, Matthias Fenchel and Peter Speier: “A Novel Method for Contact-Free Cardiac Synchronisation Using the Pilot Tone Navigator,” Proceedings of the International Society for Magnetic Resonance in Medicine (ISMRM), 2016, p. 416. The pilot tone method is further described in U.S. Pat. No. 10,222,443 (and corresponding DE 10 2015 203 385 A1) and U.S. Patent Application Publication No. 2017/0160364 (and corresponding DE 10 2015 224 162 A1), which are incorporated herein by reference. The pilot tone method describes a way of extracting the various signal components from a multi-channel pilot tone signal using a principal component analysis and/or independent component analysis, and a description of the processing of the pilot tone signal is incorporated herein by reference.

Another electromagnetic navigation method is known, for example, from E. Graesslin, G. Mends, A. Guillaume at al. “Advancements in Contact-Free Respiration Monitoring using RF Pick-up coils,” Proceedings of ISMRM (2010), p. 3045. This EMN method relies on monitoring the complex currents in the transmit RF-coils used during magnetic resonance imaging via Pick-up coils. Short rectangular monitoring pulses are integrated into the pulse sequence used for imaging. The drift of the RF-amplifiers is seen in the pick-up coils signals, and corrected by magnitude correction of the pick-up coils signals with the forward power measured between amplifier and circulator output. However, this drift correction requires further measurements in the receive path electronics.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method and a medical imaging device capable of providing quantitative motion information in order to acquire medical images with reduced motion artifacts are provided.

As another example, a novel way of characterizing and compensating a drift in a navigation signal in EMN is provided.

All features, embodiments, and advantages described with regard to one aspect are also applicable to all other aspects and categories. Embodiments and features are set out in the description and figures.

According to a first aspect, a method for generating a movement signal of an object (e.g., of a body part of a human or animal) is provided. The movement signal provides quantitative information on a movement of the object. The method includes acquiring a navigation signal from the object. The navigation signal is an electromagnetic signal that is modulated by movements of the object. A reference signal is extracted from the navigation signal. A parameter having a known time-dependency is determined from the reference signal. The navigation signal is corrected based on the parameter or the time-average of the parameter in order to reduce a signal drift in the navigation signal. The movement signal is extracted from the navigation signal.

One or more of the present embodiments have recognized that the signal drift in EMN is caused by changes in coil sensitivity, receiver gain, and signal routing attenuation. The most likely cause for these changes is the heating of the components (e.g., due to RF and gradient activity of a magnetic resonance scanner). Therefore, the signal drift is relatively slow compared to physiologic movements, such as cardiac and respiratory motion, and this opens up the possibility of using signal components having a known time-dependency (e.g., signal components related to quasi-stationary or quasi-periodic physiologic movements) for correction. The terms “motion” and “movement” are used interchangeably herein.

One or more of the present embodiments involve extracting a reference signal from the navigation signal, and determining a parameter having a known time-dependency from the reference signal. For example, the parameter may be constant in time, at least when time-averaged (e.g., over 1 to 120 seconds or 5-30 seconds). Such a parameter may be extracted from a known characteristic of the reference signal (e.g., a quasi-periodic behavior; the parameter characterizes a quasi-periodic time behavior of the reference signal). Thus, since the expected time dependency of the parameter is known, any difference between the actually observed time dependency and the known time dependency (e.g., expected time dependency) is to be due to the signal drift. Therefore, a measure for the signal drift may be determined during an examination of the human or animal and may be used to correct the signal drift in the movement signal. Thus, the parameter serves as a gain reference value. Since the reference signal and the movement signal are both extracted from the same navigation signal, the reference signal and the movement signal will be subjected at least approximately to the same signal drift.

In case a parameter or time-averaged parameter that is constant over time is determined from the reference signal, the movement signal may, for example, be multiplied with the normalized inverse of the parameter in the correction. In one embodiment, the parameter is related to the signal amplitude. The parameter may, for example, be the absolute peak-to-peak amplitude in case the reference signal or a parameter derived from the reference signal (e.g., peak-to-peak amplitude) is a signal expected to be constant over time or at least constant when averaged over a time interval that is short enough to follow the drift. In useful embodiments, the parameter is determined repeatedly (e.g., in pre-determined time intervals), and the movement signal is continuously corrected using the latest calculated parameter. The parameter may be any parameter that may be calculated from the reference signal (e.g., the absolute signal size of the signal), a slope of the reference signal, or an amplitude of an oscillation in the reference signal. If such parameters are known to be constant over time, any drift in these parameters is to be due to unwanted signal drifts in the navigation signal, and thus, may be used for correction.

The corrected movement signal may be used for navigating a medical examination (e.g., a medical image acquisition or scan from the body part going on at the same time; while the navigation signal is acquired). The image acquisition may be by any known medical imaging modality, such as CT, SPECT, PET, ultrasound, and MRI. MRI, however, provides the equipment for measuring an EMN signal. The body part may by any body part of which diagnostic images are to be acquired (e.g., head, chest, abdomen, shoulder, hip, or part of the extremities such as arms and legs). The image acquisition may be synchronized or gated by the drift-corrected movement signal.

The navigation signal may be any electromagnetic navigation (EMN) signal that is modulated by movement of the object and thus able to detect movement in a part of the human or animal body (e.g., allowing the detection of respiratory and cardiac movement), and suffering from gain drift. For example, the navigation signal is an EMN signal, in which the absolute signal size (and not only the frequency or phase of the signal) is modulated by movements of the object.

In one or more of the present embodiments, the navigation signal is an electromagnetic signal including a plurality of (e.g., several) signal components. At least some of the signal components correspond to different, independent movements of the object (e.g., the various physiologic motions such as respiration and heartbeat). The navigation signal may be acquired from a probe close to the part of the body from which the movement signal is to be generated. The probe may be a coil (e.g., a local RF coil), an observation circuit, or another electromagnetic sensor. In one or more embodiments, the probe has a plurality of (e.g., several) individual detectors, such as the various coils of multi-channel RF-coils (e.g., an array coil). In one or more embodiments, the various channels deliver linear mixtures of the individual components of the navigation signal. By principal component analysis (PCA) and/or independent component analysis (ICA), the various signal components having different characteristics in the time- and/or frequency-domain (e.g., the signal components corresponding to the different physiologic movements) may be extracted from the navigation signal.

The navigation signal may be either pulsed or continuous (e.g., a continuous-wave signal). The navigation signal may be modulated by movements of the object via various mechanisms. For example, the navigation signal detects a reflection of an electromagnetic signal in the object, or the gain, the efficiency, the degree of tuning/detuning, the phase or the loading of a coil (e.g., a radio frequency (RF) coil) placed close to the human or animal body is measured. The navigation signal may, for example, be acquired from a biomatrix respiratory sensor that is based on an oscillator circuit (e.g., built into the patient table of a medical imaging scanner). The loading of the oscillator circuit is measured continuously during a medical imaging examination, thereby generating a navigation signal that may be used in the method of one or more of the present embodiments. In another embodiment, the navigation signal is a pulsed signal and is measured by pick-up coils on the transmit RF coil, for example, as described in E. Graesslin, G. Mends, A. Guillaume, at al., “Advancements in Contact-Free Respiration Monitoring using RF Pick-up coils,” Proceedings of ISMRM (2010), p. 3045. In a further embodiment, the navigation signal is a Pilot Tone signal, as described in more detail below.

For all these techniques, a drift correction is important, since the movement desired to be detected, such as cardiac and respiratory movement, modulates the signal amplitude only to a small degree (e.g., up to 1 to 5 percent). However, if not just a movement signal allowing the detection of extreme motion states (e.g., time points of end-systole and end-diastole) is to be derived, the signal drift in the navigation signal is corrected, since otherwise, any signal drift will falsify the detected movement state. For respiratory movement, it is quite common that the extreme motion states of a particular human subject or patient will change over an examination: In the beginning, the patient may be nervous and breathe shallowly. During the examination, the patient may relax, and the breathing is deepened. Thus, the positions of the organs at the extreme motion states (e.g., end-expiration and end-inspiration) will change considerably. By detecting only the time points of such extreme motion states, and assuming corresponding positions remain constant, a good motion correction is not possible.

The reference signal may be a signal component of the navigation signal (e.g., a signal component having a certain time-dependency known a-priori). In one or more embodiments, the reference signal corresponds to a physiologic movement of the object. Further, the reference signal may have a quasi-periodic time behavior. “Quasi-periodic” may be that the movement cycles are not exact, neither in frequency nor phase, such as the physiologic motions such as heartbeat and respiration. The reference signal may be extracted, for example, by a frequency analysis if an expected frequency is within a certain known range. For example, the cardiac signal has a known quasi-periodic time-dependency and may therefore be extracted by a frequency analysis. In case the reference signal is recorded on a plurality of (e.g., several) channels, the various signal components may be extracted by principal component analysis (PCA) and/or independent component analysis (ICA) or other blind and/or semi-blind source-separation techniques. Blind/semi-blind methods generally deal with the problem of extracting components of a signal mixture with no (blind) or limited (semiblind) a-priori knowledge of the actual sources. An alternative to ICA is, for example, the SOBI algorithm.

From this reference signal, a parameter having a known time-dependency is determined, where the known time-dependency may also be that the parameter is constant or at least a time-average of the parameter is constant. Accordingly, in one or more embodiments, a reference signal is identified in the pilot tone data, with an amplitude behavior that is known and independent of the drift. From this amplitude behavior, a parameter having a known time-dependency is determined (e.g., a constant parameter such as the amplitude of a periodic or quasi-periodic oscillation in the reference signal, or the absolute signal size). In case the parameter is expected to be constant, the navigation signal may be multiplied with the inverse of the parameter in order to reduce and ideally eliminate signal drift.

Finally, the desired movement signal providing quantitative information on a movement of the object is extracted from the navigation signal, again, for example, by frequency analysis, in case the movement has a frequency in a known range, or by PCA and/or ICA. In one embodiment, the desired movement signal also corresponds to a physiologic movement of the object (e.g., a quasi-periodic movement). Since such a movement signal is extracted from the corrected navigation signal, a drift of the movement signal will be minimized. Thus, the movement signal will provide quantitative information on the movement of the object. Such movement may be any movement (e.g., respiratory, cardiac or voluntary patient movement). Accordingly, the corrected movement signal may be used for synchronizing the acquisition of a medical image data set, or in retrospective gating of a medical image data set acquired while the method of one or more of the present embodiments was carried out, in order to reduce undesired motion artifacts in the medical image data set.

According to an embodiment, the reference signal has a quasi-periodic time-dependency, and the parameter is the amplitude of the quasi-periodic signal. For example, the reference signal may be caused by a quasi-periodic physiologic motion such as the heartbeat. This choice is advantageous because a quasi-periodic signal component may be easily extracted from the navigation signal, and the amplitude of a periodic curve may easily be determined.

According to an embodiment, the known time-dependency of the parameter determined from the reference signal is that the parameter is constant over time, or at least the time-average of the parameter is constant over time. Thereby, the parameter may serve as a measure for signal drift. One or more of the present embodiments have recognized that the signal drifts in the navigation signal may be large compared to the physiologic motion signals, but are usually very slow compared to the physiologic motions, and typically evolve over several tens of seconds to minutes. Therefore, drift characterization data, such as the parameter extracted from the reference signal, may be averaged over relatively long time periods in order to increase accuracy. The average time period may, for example, be 0.5-100 seconds or 1-20 seconds.

According to one embodiment, the reference signal is the cardiac signal component, or cardiac trace (e.g., a signal related to the heart movement of the body of the human or animal). In an embodiment, the parameter extracted therefrom is the peak-to-peak amplitude between the point of maximum (e.g., end of systolic phase) and minimal (e.g., end of diastolic phase) contraction. The cardiac signal component is particularly suitable, because the cardiac signal component consists of a quasi-periodic curve with the special property that the peak-to-peak amplitude is nearly constant over time, especially when averaged over several cycles of heart movement or heartbeats (e.g., over 5-50 or 10-20 heartbeats). The reason is that the heart of a human (e.g., when at rest) tends to have a relatively constant stroke volume, when averaged over several heartbeats, even if irregularities in the frequency are common. Therefore, the amplitude of the cardiac signal component (e.g., the peak-to-peak amplitude) may serve, after sufficient averaging, as a measure of the receiver gain. Thus, the amplitude of the cardiac signal component may be used to correct the gain (e.g., for each channel). Interestingly, the cardiac signal is likely to be visible not only if the body part is the heart, but also in all other parts of the body (e.g., the head, abdomen, and even the extremities) due to pulsatile flow expanding the blood vessels in all parts of the body.

In practice, the cardiac signal component may still be contaminated with respiratory residues. However, these signal contributions may be separated before the analysis (e.g., before determining the parameter, such as peak-to-peak amplitude, based on the lower frequency content).

According to an embodiment, the navigation signal is acquired continuously over the duration of the medical examination or scan, and the parameter or the time-average of the parameter of the reference signal is determined repeatedly during the medical examination. This allows continued correction of the signal drift. Thus, the method may be performed during a medical examination (e.g., a medical imaging procedure on the body part). One or more of the present embodiments are applicable in magnetic resonance imaging, but may also be used during a CT, X-ray, PET, or single photon emission computer tomography (SPECT) examination (e.g., any medical imaging procedure having acquisition times longer than one breath-hold).

In case the parameter used as gain reference value is related to a quasi-periodic time-dependency of the reference signal, the parameter may be determined at each period of the quasi-periodic signal (e.g., averaged over several periods), and the following portion of the navigation signal is corrected based on this parameter. In the next periods of the reference signal, a new time-average parameter is determined, and then the new parameter is used for the correction. Thereby, the parameter or time-averaged parameter is determined repeatedly during the medical examination. In other embodiments, the parameter is determined afresh again in specified time intervals (e.g., every 1-3 minutes) and used for correction during the next time interval.

According to an embodiment, the parameter is normalized. Thus, when the parameter is first determined, the parameter is set to the value one. When the parameter or the time-averaged parameter is determined again, the same normalization factor is used, so that any variation or drift in the parameter will become apparent in that the parameter does not have the value of one any more. Thereby, the calculation of the corrected movement signal is simplified.

According to an embodiment, the movement signal finally extracted from the navigation signal provides quantitative information on a quasi-periodic physiologic movement of the human or animal (e.g., on a respiratory movement). This application of the present embodiments is advantageous, since the respiratory movement is relatively great, causing displacements by several centimeters. As mentioned before, the respiratory movement is not particularly regular, and various breaths during examination may be deep or shallow. Accordingly, it is important to have quantitative information (e.g., a movement signal the curve of which is directly related to the position of the moving body part at each time point, such as by a linear correlation). Thereby, the position of each organ may be exactly determined on an image acquired by the medical examination.

According to an embodiment, the navigation signal is acquired on a plurality of (e.g., several) signal channels (e.g., by a multi-channel RF coil for magnetic resonance imaging). Thereby, the navigation signal has a plurality of (e.g., several) signal channels. By combining the various channels in a certain way, the different signal components having specific characteristics, such as the cardiac signal component and the respiratory signal component, may be extracted. In one or more embodiments, the channel combination coefficients for each desired signal component is determined from a calibration portion or calibration phase of the movement signal, acquired, for example, during 1 to 60 seconds before or at the beginning of a medical examination (e.g., by PCA and/or ICA), whereby a demixing matrix is determined, as described below.

The correction does not change the channel combination coefficients significantly because gain variations are slow and small (e.g., within a few percent), and the modulation depth of the navigation signal (e.g., a pilot tone signal due to the much faster physiologic processes; the net navigation signal) is on the order of a few percent as well. Therefore, the first order effect of a channel-wise gain variation is a slowly varying offset in the channel combined pilot tone component. Therefore, instead of applying the correction for each channel individually, the correction may be applied to the combined signal instead (e.g., to the extractive movement signal).

Thus, in an embodiment, the correcting of the navigation signal and the extracting of the movement signal of the method of one or more of the present embodiments may be interchanged. Thus, the movement signal is first extracted from the uncorrected navigation signal. The correction based on the parameter or the time-average of the parameter is then carried out on the extracted movement signal in order to compensate a signal drift in the movement signal. If the movement signal is a linear combination of the various signal components or channels of the navigation signal, it makes no difference whether the correction is applied to each channel individually or to the combined signal (e.g., the extracted movement signal) instead.

According to an embodiment, the reference signal and the movement signal (e.g., reference signal component and movement signal component) are extracted from the navigation signal by a PCA, ICA, and/or a frequency analysis. This is based on the fact that the various signal components have different characteristics (e.g., different frequencies that are independent from one another). Therefore, the various signal components may be separated from each other.

According to an embodiment, the navigation signal is a pilot tone (PT) signal, where the pilot tone signal is generated by: emitting a radio frequency (RF) signal outside the bandwidth of an MR signal acquired during magnetic resonance imaging of the part of the human or animal body, and recording the radio frequency signal, modulated by a movement of the human or animal body, by an RF coil (e.g., a multi-channel RF coil for magnetic resonance imaging). The recorded radio frequency signal is the navigation signal. In one embodiment, the RF signal is a coherent or continuous frequency signal generated by an independent continuous-wave RF source outside the receive bandwidth of the actually scanned MR field of view, but within the range of the oversampling bandwidth acquired during every readout. The RF signal may not be pulsed, but at least quasi-continuous. Therefore, a corresponding frequency bandwidth is very narrow and may be selected outside the bandwidth of a MR signal acquired during MR imaging (e.g., about 20 to 200 kHz away from the center frequency of the RF coil).

The pilot tone (PT) is useful in that the PT is outside the receive bandwidth of the magnetic resonance image, and therefore, does not interfere with the magnetic resonance (MR) signal. Further, the pilot tone signal is acquired independent of the imaging sequence and may therefore be used with arbitrary imaging sequences.

A method for reliably extracting the cardiac movement signal from the PT signal is described in EP 17179814.3, and the method is also applicable for extracting the respiratory movement signal. For example, this application describes steps of providing a pilot tone signal acquired from the body part by a magnetic resonance receiver coil arrangement including a plurality of (e.g., several channels), where the pilot tone signal includes a plurality of (e.g., several) signal components associated with the several channels. From a calibration portion of the pilot tone signal, a demixing matrix is calculated by an independent component analysis (ICA) algorithm, where the demixing matrix calculates the independent components from the several signal components. The independent component(s) corresponding to at least one particular movement type (e.g., the cardiac movement) is selected, and the demixing matrix is applied to the further portions of the Pilot Tone signal to obtain at least one movement signal representing one particular movement type (e.g., the cardiac movement).

The description and examples of these acts in U.S. Patent Application Publication No. 2018/0353140 (and corresponding EP 17179814.3) may be also used in the present embodiments and is incorporated herein by reference.

The “calibration portion” of the pilot tone signal may be a short portion covering only a few (e.g., 1 to 20) heart beats acquired before or at the beginning of the scan of medical data. From this calibration portion, a demixing matrix is determined (e.g., by first determining the principle components through PCA, and then identifying the physiologic components by ICA). Prior to the calculation of the demixing matrix, the pilot tone signal may be processed (e.g., pre-processed by down-sampling to a new sampling frequency that is sufficient to capture cardiac dynamics; to 50 to 300 Hz). To avoid aliasing of high-frequency noise, the signal may be low pass filtered prior to down sampling. In one or more embodiments, the phases of all channels are then normalized to a reference phase of a selected channel, and only relative phase offsets to this reference are further considered. In order to reduce the complexity of the ICA problem, further pre-processing acts may be performed before the calculation of the demixing matrix, or before the ICA algorithm. These may include centering (e.g., subtracting the mean so that the resulting signals are zero-mean), whitening/sphering to provide that all signals have unit variance and are uncorrelated (e.g., for a given (n×m) matrix x), where n is the number of samples and m is the number of channels, Cov(x)=E{xxT}=I, with I the identity matrix, and dimensionality reduction.

After such pre-processing, ICA is used to extract a plurality of (e.g., several) independent components from the calibration portion of the pilot tone signal. Once the independent component(s) corresponding to the desired movement types have been selected, a demixing matrix or vector that extracts these movement type(s) is applied to the further portions of the pilot tone signal (e.g., in real time). When several movement types are to be extracted, applying the demixing matrix results in several movement signals, each representing one particular movement type.

According to an embodiment, a pilot tone signal component of a physiological signal with a known characteristic, such as the constant amplitude of the quasi-periodic cardiac trace, is used as gain reference. This enables drifts to be compensated in the receive gain of the individual channels of the electromagnetic navigation signal. Thereby, quantitative movement information may be extracted from the navigation signal (e.g., on the respiratory movement state).

One or more of the present embodiments are also directed to a method for generating a medical image data set of an object undergoing a movement (e.g., a part of the human or animal body, such as any body part of which images are to be acquired, such as the head, the chest, the abdomen, the shoulder, the hip, or any extremity). Placing the object in the medical imaging device provides that, for example, a human subject or patient is placed on a patient table and shifted into the sensitive region of the medical imaging device (e.g., the bore of an MR scanner).

The movement signal is used during or after image acquisition, either for synchronizing the MR image sequence to the movement signal (e.g., to the respiratory movement of the part of the human or animal body), or for retrospectively gating the MR image data acquired, so that medical images may be reconstructed without motion artifacts. By “synchronizing or gating”, all methods that use information on the movement state of an object (e.g., an object undergoing a quasi-periodic movement) to record an image without or at least reduced motion artifacts may be provided. This may be done, for example, by synchronizing or timing the acquisition such that image date acquisition takes place at the desired motion states. These may be either at times when there is no motion, or care is taken to a acquire data at specific motion states of the object, so that the image data set includes a time-series of images that may be viewed in sequence to analyze the movement (e.g., cine-mode). By “gating”, the present movement state is recorded together with the image data recorded at the time, so that the data may be reconstructed retrospectively by rearranging the data correctly (e.g., all image data corresponding to one movement state are reconstructed into one image).

The image data set may be a two-dimensional (2D), three-dimensional (3D), or four-dimensional (4D) image data set, where the fourth dimension is time. Such image data set may include an array of data points (e.g., including grey level information) and may include a header containing information on the time of image. The image data set may, for example, be in DICOM standard.

One or more of the present embodiments are also directed to a medical imaging device (e.g., a magnetic resonance imaging device (e.g., MR scanner)) adapted to perform the method of one or more of the present embodiments. The MR scanner includes a magnet for generating the main magnetic field, gradient coils, as well as an RF coil for acquiring the navigation signal from the object. In case of pilot tone navigation, the pilot tone signal may be emitted by an external frequency source that is placed into the bore of the main magnet. However, the pilot tone signal may also be emitted by the body coil. The medical imaging device may also be an ultra sound device, PET, SPECT, or CT device.

One or more of the present embodiments are also directed to a computer program or computer program product (e.g., a non-transitory computer-readable storage medium) including software code portions that induce a processor (e.g., a processor controlling a medical imaging device such as an MR scanner) to perform the method of one or more of the present embodiments when the software code portions are executed on the processor. The processor may be part of a workstation related to a medical imaging device, or the processor may be part of a standalone computer, cloud computer, server, tablet computer, or any mobile or portable device such as a laptop.

According to a further aspect, one or more of the present embodiments are directed to a digital storage medium on which the computer program or computer program product is stored. The digital storage medium may be part of a computer, such as a hard disc, solid state disc, etc., where the computer may also include the processor described herein. Further, the digital storage medium may be a cloud storage, the storage of a medical imaging device, or any kind of portable storage such as a USB-stick, CD-ROM, SD-card, etc.

### BRIEF DESCRIPTION OF THE DRAWINGS

FIG. 1 shows a magnetic resonance scanner according to an embodiment in a schematic view;

FIG. 2 shows a graph of an example pilot tone signal, and a respiratory signal component and cardiac signal component extracted therefrom, in signal amplitude in arbitrary units versus time in seconds;

FIG. 3 shows a graph of a filtered cardiac signal component during one heart cycle;

FIG. 4 shows graphs of a simulated respiratory signal (top) and simulated cardiac signal (bottom), in signal amplitude in arbitrary units versus time in seconds; and

FIG. 5 shows a schematic flow diagram of an embodiment of a method.

### DETAILED DESCRIPTION

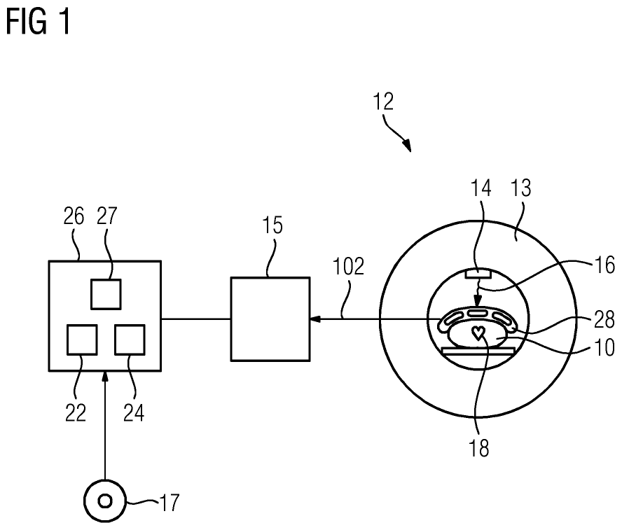

With reference to FIG. 1, a magnetic resonance (MR) scanner 12 includes a main magnet 13, with a human subject 10 or patient placed in a bore of the main magnet 13. A receiver coil arrangement 28 (e.g., a local coil such as a head-coil or chest-array-coil) is placed close to the body part from which images are to be acquired. The human subject 10 is subject to various movements (e.g., the two physiologic, quasi-periodic movements of respiratory motion and the motion of the heart 18). The MR-scanner 12 includes further components not shown herein, such as gradient coils for generating gradient fields, as well as, for example, a body coil. The body coil may be used as an RF transmitter for transmitting the RF-pulses for MR image acquisition, and possibly the radio frequency signal outside the MR receive bandwidth, used for generating the pilot tone signal 16. In the embodiment shown, the pilot tone signal 16 is emitted by a separate RF source 14, which is positioned inside the bore of the main magnet 13. In one embodiment, such a separate RF source 14 may be strapped to the local coil 28. The pilot tone signal 16 is modulated by the movement of the heart 18 and further movements of the human subject 10. The modulated pilot tone signal together with the MR signal 102 is received by the receiver coil arrangement 28 and is further transmitted to a receiver 15, which includes electronic components as known in the art (e.g., a pre-amplifier and amplifier), as well as other electronic components such as filters. The receiver 15 is a likely contributor to gain changes creating large drifts in the net pilot tone signal. The receiver 15 is connected to a computer device 26 including a control unit 24, which controls the activity of the MR-scanner 12 (e.g., the image data acquisition). The MR signal 102, including the PT signal, is received and amplified by the receiver 15 and further processed in the control unit 24, which may include a processor, and/or the computer device 26. The computer device 26 may also include a digital storage medium 22 and a user-interface 27 including, for example, a display, a keyboard, a mouse, a touchscreen, or such like. A compact disk 17 may include a computer program that, when loaded into the computer device 26, configures the MR-scanner 12 to perform a method according to one or more of the present embodiments.

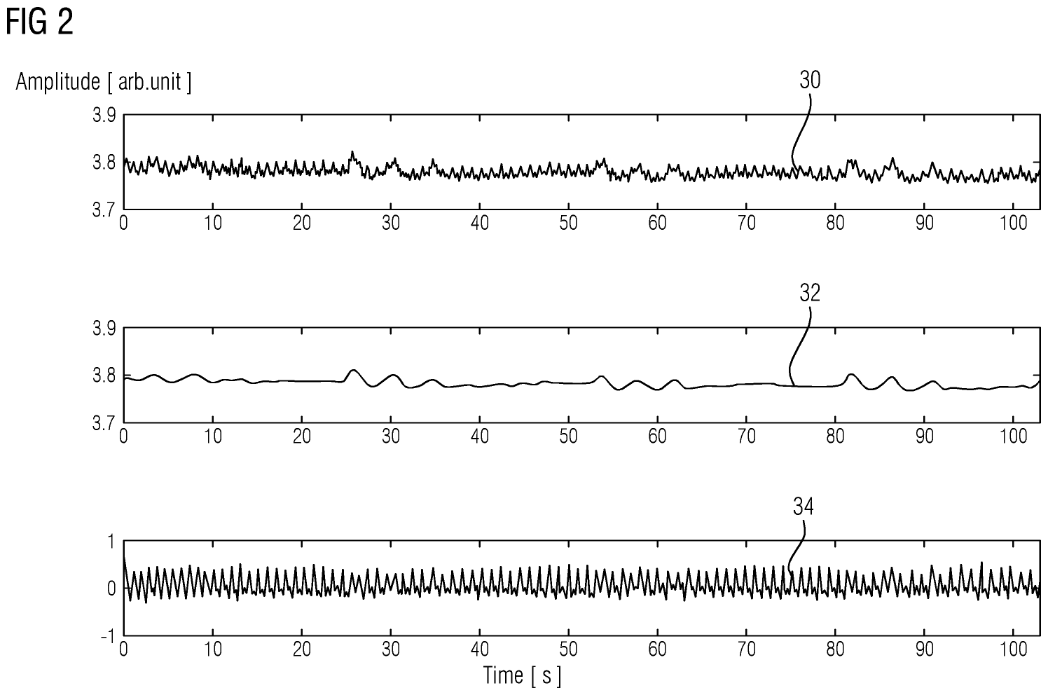

FIG. 2 illustrates the various signal components of the pilot tone signal (e.g., a navigation signal). The navigation signal may be acquired on a plurality of (e.g., several) channels. Each channel of the plurality of channels may correspond to one coil element of the RF coil 28. Thus, the navigation signal includes a plurality of (e.g., several) signal components or channels. The upper trace 30 shows one channel of the navigation signal. This signal 30 is a super position of various signal elements having different characteristics and frequencies. These various signal components may be extracted from the navigation signal by frequency analysis, or in the case of a signal having several signal channels, by PCA and/or ICA. Thereby, the various signal components may be extracted. For example, trace 32 shows a respiratory signal acquired during instructed breathing. The subject is told to take three deep breaths, interrupted by a breath hold. Trace 32 demonstrates very well the fact that the respiratory movement, although approximately periodic (e.g., quasi-periodic), is highly irregular: some breaths are deep, others are shallow. Therefore, if one wants to extract quantitative information, for example, from the absolute signal size or amplitude of the respiratory signal, drift is to be prevented.

Trace 34 shows a cardiac signal extracted from the navigation signal 30 and a distinct periodic behavior with the frequency of about 1 s⁻¹ corresponding to the heartbeat. The amplitude of the oscillation of the cardiac signal 34 is variable from heartbeat to heartbeat, but is roughly constant when averaged over, for example, more than 10 heartbeats. Thus, the amplitude may be used, after sufficient averaging, as a measure of the receiver gain for each channel and may be used to correct the gain for each channel.

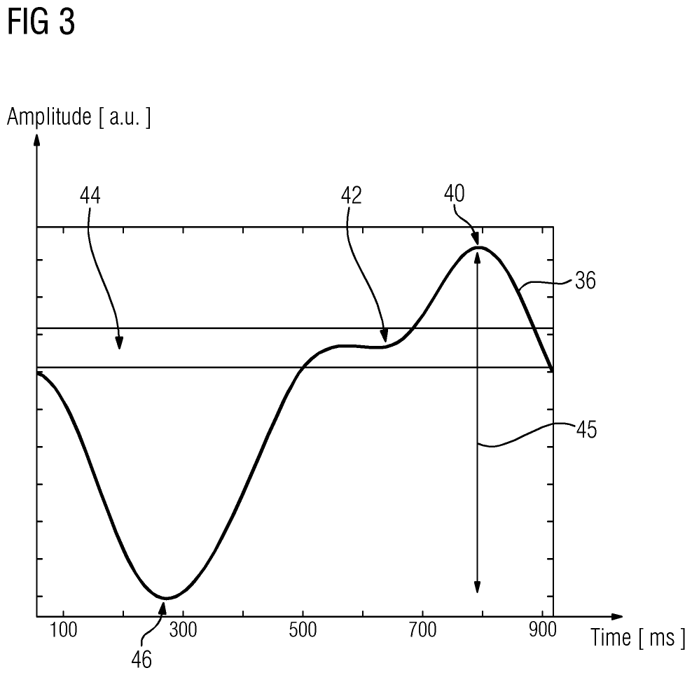

The cardiac signal component 36 of one cardiac cycle is shown in magnification in FIG. 3. The cardiac signal component 36 shown, also referred to as cardiac trace, has been filtered (e.g., by a switched Kalman filter based on a model generated by analysis of the cardiac component acquired during a calibration phase). This filtered cardiac trace 36 is shown in a plot of amplitude in arbitrary units versus time. From the filtered cardiac trace 36, the following points of interest may be determined: The minimum 46 of the cardiac component trace 36 indicates end-systole (e.g., the maximum contraction and resting phase). The maximum 40 of the cardiac component indicates end-diastole (e.g., the physiological phase of maximum expansion of the heart during the resting phase). The plateau 42 may be associated with the mid-diastolic phase, in which the ventricle is relaxed (e.g., a resting phase). The area 44 indicates the signal level for R-wave occurrence and may be used in a threshold trigger. From the cardiac trace 36, the first and/or second derivative may also be derived. The minimum of the first derivative of the cardiac trace indicates times of maximum velocity. The difference in amplitude between minimum 46 and maximum 40 is the signal difference 45 between the diastolic and the systolic phase of the heart and may be used as a gain reference, especially when averaged over a plurality of (e.g., several) heart-cycles. For example, the respiratory signal component 32 may be divided by the averaged signal difference 45, so that any signal drift occurring in both signals is eliminated.

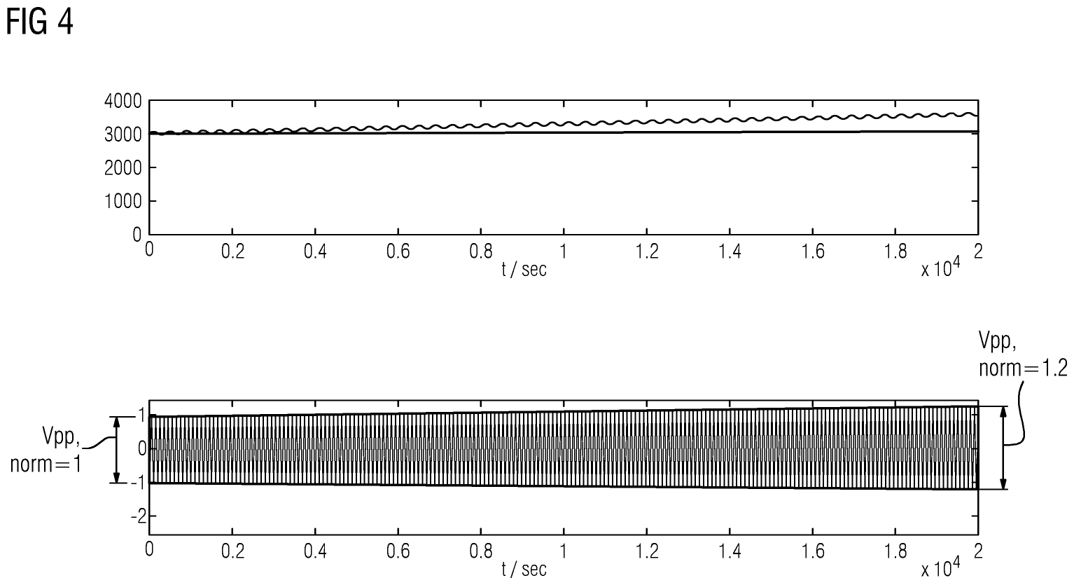

FIG. 4 shows simulated respiratory (top) and cardiac (bottom) signals in order to demonstrate the effect of the gain drift: In the simulated respiratory signal in the top graph, the offset (e.g., the absolute signal amplitude) is large (e.g., 3000 a.u.) compared to the respiratory signal having a modulation depth of about 1.6%. An assumed slow, linear gain drift from gain=1 to gain=1.2 is simulated. Since the absolute amplitude of the respiratory signal is much higher than the modulation depth of the motion signal, gain variation presents primarily as drift. The bottom graph shows a simulated cardiac trace, in which the offset (e.g., 5000 a.u.) has been removed for better visibility. Since it may be assumed that the peak-to-peak amplitude is constant over time, any change in observed peak-to-peak signal amplitude Vpp is to be due to gain changes. Thus, the gain variation may be approximated by continuously measuring the peak-to-peak amplitude. Vpp is normalized at the beginning to Vpp,norm=1 to allow for simple division of the, for example, respiratory signal with Vpp,norm. At the end of the observed time period, Vpp,norm has risen from 1.0 to 1.2.

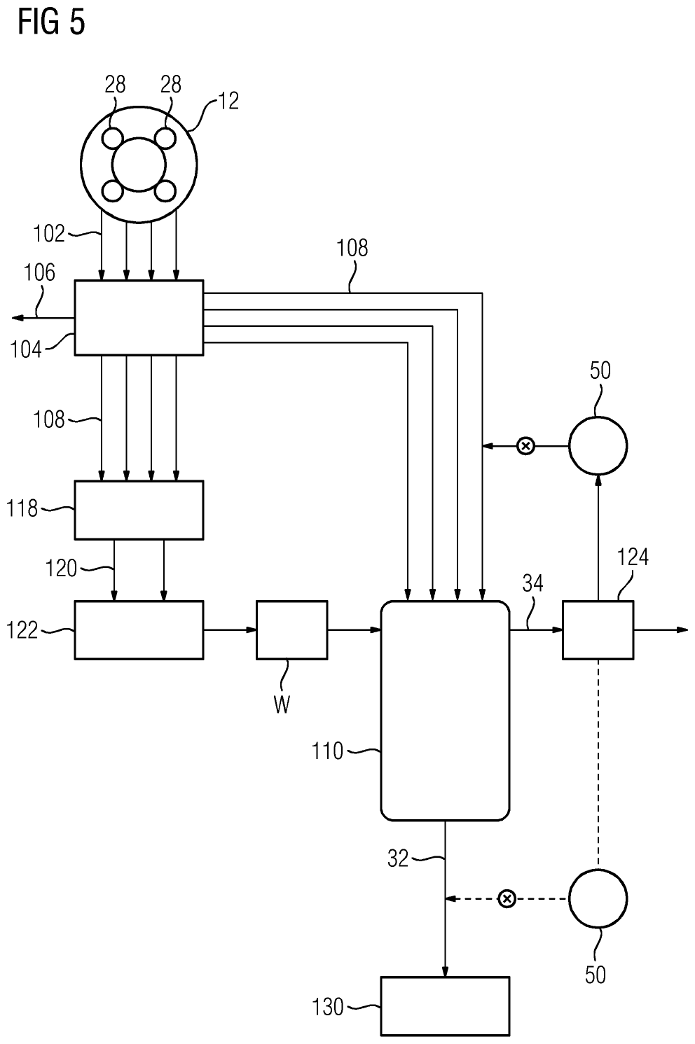

FIG. 5 gives an overview of the method according to one or more of the present embodiments. The MR-scanner 12 includes a receiver coil arrangement 28 with, in this schematic example, four coils/channels. When the acquisition of the navigation signal and MR signal starts, the RF coil acquires a signal 102 having four signal components. In a separation act 104, the navigation signal 108 (e.g., a complex pilot tone signal) is separated from the MR data 106. The MR data 106 is further possessed to produce MR image data, as known in the art. In act 104, the absolute frequency of the navigation signal may be determined.

The pilot tone signal 108 including the four channels or signal components is optionally subjected to further processing acts, such as pre-processing by low-pass or band-pass filtering and centering. The pre-processed signal may further be subjected to a normalization act, in which the phases of all channels are normalized to a reference phase.

The optionally normalized, complex pilot tone signals 108 are then further processed in act 118 to separate the various movement types, by which the pilot tone signal is modulated. According to an embodiment, this is done first by principle component analysis, in which the largest principle components are extracted. These are then subjected to independent component analysis (ICA). Through the ICA, the different signal components 120 corresponding to the different movement types are separated. Thereby, a reduction in dimensionality occurs (e.g., four components 108 are reduced to two components 120 representing respiratory motion and cardiac motion). To identify the cardiac signal component from the independent components 120, for example, the signal energy in the cardiac motion band may be calculated for each independent component, compared to the signal energy in other frequency bands, and the signal component with the highest relative signal energy in frequency band corresponding to cardiac motion is selected. Alternatively, the degree of correlation of each signal component with a typical cardiac trace may be calculated. Similarly, the single component corresponding to the respiratory movement may be calculated. Once the independent component has been correctly identified, a demixing matrix W may be calculated (e.g., automatically). The demixing matrix W may correspond to a linear combination of the signal components 108 of the several receiver channels 28.

Acts 118 and 122 may be carried out on a calibration portion of the navigation signal at the beginning of the medical examination. During the calibration phase, the coil combination corresponding to the reference signal (e.g., cardiac signal component) is detected by the usual processes (e.g., ICA or frequency analysis).

The demixing matrix W is then stored and used during the main part of the medical examination. For example, the demixing matrix W is applied to the incoming further pilot tone signal 108, which is multiplied with the demixing matrix W in act 110 to obtain at least one selected independent component (e.g., the cardiac signal component 34 and the respiratory signal component 32). The cardiac trace 34 is analyzed in act 124, either continuously or in suitable intervals during the image acquisition. In act 124, the signal difference 45 between the diastolic and systolic phase is determined and averaged over a plurality of (e.g., several) cycles of heart movement. This is used as the gain reference parameter 50, which may be used to correct the navigation signal in two alternative ways: As shown in unbroken lines, the gain reference parameter 50 may be used to correct the pilot tone signal 108 (e.g., the uncombined pilot tone data before applying the demixing matrix in act 110 such as by multiplying with its inverse). Alternatively, as shown in dashed lines, instead of applying the correction for each channel individually, the correction may be applied to the combined respiratory signal component 32 instead (e.g., after the movement signal has been extracted from the navigation signal). In either case, the demixing matrix W is also applied to the corrected or uncorrected pilot tone signal 108 in act 110 in order to separate the respiratory signal component 32, which may be used as a movement signal to synchronize the MR acquisition in real time in act 130. In useful embodiments, the reference value 50 is updated, for example, every 30-300 seconds. One or more of the present embodiments have recognized that the gain drifts are very slow compared to the physiologic motions. Thus, it drift characterization data (e.g., the signal difference between diastolic and systolic phase in the cardiac trace) may be averaged over long time periods to increase accuracy.

In other words, during the measurement, such as MR data acquisition, the reference signal is continuously detected together with the desired physiologic components (e.g., respiration). The reference signal is normalized to yield a normalized preference signal (e.g., the reference signal is corrected based on the expected characteristic). In case of the cardiac trace, the difference between diastolic and systolic amplitude is determined.

Time-averaging of the normalized reference signal eliminates potential residual respiratory contamination and increases the signal quality. The time resolution may be small compared to physiologic variations (e.g., heartbeat duration and potentially respiratory duration), but high enough to follow the gain drift.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for generating a movement signal of an object, wherein the movement signal provides quantitative information on a movement of the object, the method comprising:
acquiring a navigation signal from the object, wherein the navigation signal is an electromagnetic signal that is modulated by movements of the object;
extracting a reference signal from the navigation signal;
determining a parameter having a known time-dependency from the reference signal;
reducing a signal drift in the navigation signal, the reducing of the signal drift comprising correcting the navigation signal based on the parameter or a time-average of the parameter; and
extracting the movement signal from the navigation signal.

2. The method of claim 1, wherein the reference signal has a quasi-periodic time dependency, and the parameter is an amplitude of the quasi-periodic time dependency.

3. The method of claim 1, wherein the known time-dependency is that the parameter is constant over time or the time-average of the parameter is constant over time.

4. The method of claim 1, wherein the reference signal is a cardiac signal related to a heart movement of the body of a human or an animal, and the parameter is a peak-to-peak signal amplitude between a point of maximum contraction of the heart and a point of minimum contraction of the heart.

5. The method of claim 4, wherein the parameter is the peak-to-peak signal amplitude between the point of maximum contraction of the heart and the point of minimum contraction of the heart, averaged over a plurality of cycles of heart movement.

6. The method of claim 1, wherein acquiring the navigation signal comprises acquiring the navigation signal continuously over a duration of a medical examination, and
wherein determining the parameter comprises determining the parameter or the time-average of the parameter of the reference signal repeatedly during the medical examination.

7. The method of claim 1, wherein the movement signal provides quantitative information on a respiratory movement of a human or an animal.

8. The method of claim 1, wherein acquiring the navigation signal comprises acquiring the navigation signal on a plurality of signal channels.

9. The method of claim 8, wherein the navigation signal is acquired on the plurality of signal channels by a multi-channel radio-frequency (RF) coil for magnetic resonance imaging.

10. The method of claim 1, wherein the extracting of the movement signal is performed before the reducing of the signal drift in the navigation signal, such that the movement signal is extracted from the navigation signal before the correction of the navigation signal, and
wherein correcting the navigation signal based on the parameter or the time-average of the parameter comprises correcting the extracted movement signal, such that a signal drift in the movement signal is reduced.

11. The method of claim 1, wherein the reference signal and the movement signal are extracted from the navigation signal by a principal component analysis or an independent component analysis, a frequency analysis, or a combination thereof.

12. The method of claim 1, wherein the navigation signal is a pilot tone signal, wherein the method further comprising generating the pilot tone signal, generating the pilot tone signal comprising:
emitting a radio frequency signal outside a bandwidth of a magnetic resonance (MR) signal acquired during magnetic resonance imaging of the object, the object being a body part of a human or an animal body; and
recording the radio frequency signal, modulated by a movement of the human or the animal body, by a radio frequency (RF) coil.

13. The method of claim 12, wherein the RF coil is a multi-channel RF coil for magnetic resonance imaging

14. A method for generating a medical image data set of an object undergoing a movement, the method comprising:
placing the object in a medical imaging device;
generating a movement signal of the object, the generating of the movement signal of the object comprising:
acquiring a navigation signal from the object, wherein the navigation signal is an electromagnetic signal, a amplitude of the electromagnetic signal being modulated by movements of the object;
extracting a reference signal from the navigation signal,
determining a parameter having a known time-dependency from the reference signal;
reducing a signal drift in the navigation signal, the reducing of the signal drift comprising correcting the navigation signal based on the parameter or a time-average of the parameter; and
extracting the movement signal from the navigation signal, wherein the movement signal provides quantitative information on a quasi-periodic movement of the object; and
acquiring a medical image data set from the object, the acquiring of the medical image data comprising synchronizing or gating the acquiring of the medical image data using the movement signal, such that undesired motion artifacts in the medical image dataset are reduced.

15. The method of claim 14, wherein the medical imaging device is a magnetic resonance (MR) scanner, and the medical image dataset is a magnetic resonance image dataset.

16. The method of claim 15, wherein the navigation signal is a pilot tone signal recorded by a multi-channel RF coil,
wherein the navigation signal is acquired on a plurality of signal channels, and
wherein the method further comprises determining a channel combination corresponding to the reference signal, the movement signal, or the reference signal and the movement signal during a calibration phase before or at a beginning of the acquisition of the medical image data set, and using the channel combination in extracting the reference signal from the navigation signal, extracting the movement signal from the navigation signal, or extracting the reference signal from the navigation signal and extracting the movement signal from the navigation signal.

17. A medical imaging device comprising:
a radio frequency (RF) coil configured to acquire a navigation signal from an object, wherein the navigation signal is an electromagnetic signal, an amplitude of the electromagnetic signal being modulated by movements of the object,
wherein the medical imaging device is configured to:
extract a reference signal from the navigation signal;
determine a parameter having a known time-dependency from the reference signal;
correct the navigation signal based on the parameter or a time-average of the parameter;
extract a movement signal from the navigation signal, wherein the movement signal provides quantitative information on a quasi-periodic movement of the object; and
acquire a medical image data set from the object, the acquisition of the medical image data set comprising synchronization or gate of the acquisition of the medical image data set using the movement signal, such that undesired motion artifacts in the medical image dataset are reduced.

18. In a non-transitory computer-readable storage medium that stores instructions executable by a processor to generate a movement signal of an object, wherein the movement signal provides quantitative information on a movement of the object, the instructions comprising:
acquiring a navigation signal from the object, wherein the navigation signal is an electromagnetic signal that is modulated by movements of the object;
extracting a reference signal from the navigation signal;
determining a parameter having a known time-dependency from the reference signal;
reducing a signal drift in the navigation signal, the reducing of the signal drift comprising correcting the navigation signal based on the parameter or a time-average of the parameter; and
extracting the movement signal from the navigation signal.
